(19) United States
(12) Patent Application Publication
(10) Pub. No.: US 2008/0207503 A1
(43) Pub. Date: Aug. 28, 2008

(54) Composition and Treatment Methods for Coronary Artery Disease

(76) Inventors: Byung-Hong Chung, Birmingham, AL (US); Byung Hui Cho, Champaign, IL (US)

Correspondence Address: BRADLEY ARANT ROSE & WHITE, LLP;INTELLECTUAL PROPERTY DEPARTMENT-NWJ, 1819 FIFTH AVENUE NORTH, BIRMINGHAM, AL, 35203-2104, US

(21) Appl. No.: 11/722,664

(22) PCT Filed: Dec. 22, 2005
(86) PCT No.: PCT/US05/46591

§ 371 Date: Jun. 22, 2007

Related U.S. Application Data
(60) Provisional application No. 60/693,011, filed on Jun. 22, 2005.

(51) Int. Cl. A61K 38/12; A61K 31/685; A61P 9/10

(52) U.S. Cl. 514/12; 514/78

(57) ABSTRACT

The present disclosure demonstrates that cholesterol-free discoidal reconstituted HDL (R-HDL), phosphatidyl-choline (PC) and PC liposomes effectively released cholesterol from ICP. Native HDL and its apolipoproteins were not able to release cholesterol from ICP. The release of ICP cholesterol by R-HDL was dose-dependent and accompanied by the transfer of >8× more PC in the reverse direction (i.e., from R-HDL to ICP), resulting in a marked enrichment of ICP with PC. The enrichment of ICP with PC resulted in the dissolution of cholesterol crystals on ICP and allowed the removal of ICP cholesterol by apo HDL and plasma. The present disclosure provides a method of treatment for removal of cholesterol from ICP in vivo and compositions for use in such method of treatment. Such methods may be used in the treatment and/or prevention of atherosclerosis, coronary artery disease, and related disease states and conditions.

### STATEMENT REGARDING GOVERNMENT SUPPORT

This study was supported by US Public Health Service Grants (NIH HL 60936, HL 50719 and EY 06109). As such the government may have certain rights in this disclosure.

### FIELD OF USE

The present disclosure relates to treatment methods and compositions for the treatment of atherosclerosis, coronary artery disease and related disease states and conditions.

### BACKGROUND

In numerous epidemiological and clinical studies, plasma levels of high density lipoproteins (HDL) and apo A-I (apolipoprotein A1) correlated inversely with the incidence of coronary artery disease (CAD) (1-3). This protective effect is usually attributed to HDL's role in removing cholesterol from atherosclerotic lesions and transporting it to the liver, a process called reverse cholesterol transport (RCT) (4). In vitro cell culture studies have shown that native HDL or its lipid-free apolipoproteins are able to promote the efflux of cellular cholesterol via a non-specific aqueous diffusion process (5-6), mediation by specific scavenger receptor class B type I (SR-BI) receptors on cell surfaces (7, 8), and/or microsolubilization of plasma membrane phospholipids (PL) and cholesterol, with the participation of ATP-binding cassette transporter A1 (ABCA1) (9-11). Cholesterol deposits are an essential characteristic of human atherosclerotic lesions, but the deposit of cholesterol on atherosclerotic lesions differs from that on membranes of cultured cells. In early atherosclerotic lesions, cholesterol accumulates on cells, as intracellular lipid droplets in foam cells, and as extracellular lipoprotein- and liposome-like particles (12-18). As lesions progress, foam cells die, leading to an increasingly necrotic plaque core (12, 13, 17, 18). Thus, plaque cholesterol composition is complex, containing living and dead cells, cellular debris, and extracellular particles including crystalline cholesterol (12-18). It is not clear whether HDL and its apolipoproteins can release cholesterol in advanced atherosclerotic lesions containing dead foam cells and extracellular cholesterol deposits by the same mechanisms involved in the release of cholesterol from cultured cells. It is probable that SR-B1 receptor and ABCA1 transporter may not have an important role in removing cholesterol from advanced atherosclerotic lesions containing dead foam cells and/or extracellular deposits because activities of the SR-B1 receptor and ABCA1 transporter would not be preserved.

In humans and animal models, advanced atherosclerotic lesions contain crystallized cholesterol and are highly enriched in free cholesterol (FC) and spningomyelin (SPM) (12, 15, 19). SPM-enrichment stabilizes cholesterol on cell membranes, and high FC levels may protect the dissolution of lipids in the advanced lesions by HDL and apo A-I (16). Further, the turnover of cholesterol on atherosclerotic plaques in humans is much slower than that in other tissues due to change in their physical state (20, 21), suggesting that the exchange rate of cholesterol between plaques and blood is low. For these reasons, the mechanisms and kinetics of HDL- and apolipoprotein-mediated cholesterol removal from cultured cells may not reflect those of HDL-mediated cholesterol removal from advanced atherosclerotic lesions. Indeed, early studies suggested that a fundamentally different process occurs in intima, as pure cholesterol crystals incubated with HDL form liposomes containing PL derived from HDL (22, 23).

In the present disclosure, the potential of HDL to ameliorate atherosclerotic plaques in vivo was examined. Furthermore, the ability of native HDL, lipid-free HDL apolipoproteins (apo HDL), cholesterol-free discoidal reconstituted HDL (R-HDL) comprised of apo HDL and phosphatidylcholine (PC) and PC liposomes to release cholesterol from cholesterol-rich insoluble components of plaques (ICP) isolated from atherosclerotic human aorta was examined was examined. Isolated ICP used in the present disclosure had a FC to PL mass ratio (0.8-3.1) and a SPM to PC mass ratio (1.2-4.2) that exceeded those of plasma membranes of cultured cells. Surprisingly, the present disclosure demonstrates that native HDL and its apolipoproteins were not able to release cholesterol from ICP. However, R-HDL and PC liposomes effectively released cholesterol from ICP. The release of ICP cholesterol by R-HDL was dose-dependent and accompanied by the transfer of >8× more PC in the reverse direction (i.e., from R-HDL to ICP), resulting in a marked enrichment of ICP with PC. Compared to R-HDL, PC liposomes were significantly less effective in releasing cholesterol from ICP but were somewhat more effective in enriching ICP with PC. Native HDL was minimally effective in enriching ICP with PC, but became effective after prior in vitro enrichment of HDL with PC from multilamellar PC liposomes. The enrichment of ICP with PC resulted in the dissolution of cholesterol crystals on ICP and allowed the removal of ICP cholesterol by apo HDL or plasma. The present disclosure shows that removal of cholesterol from ICP in vivo is possible through a change in the level, composition, and physical state of ICP lipids mediated by PC enrichment. Therefore, the present disclosure provides methods of treatment for removal of cholesterol from atherosclerotic plaques in vivo and compositions for use in such method of treatment. Such methods and composition have been previously lacking and unappreciated in the art.

### BRIEF DESCRIPTION OF THE FIGURES

FIG. 1A shows the levels of ICP cholesterol released into soluble fractions and cholesterol on HDL incubated without ICP following incubation of ICP (0.5 mg FC) with TBS and TBS containing apo HDL, R-HDL, or HDL at the mass ratio of ICP FC to PL on HDL and R-HDL of 1:10, or at the mass ratio of ICP FC to apo HDL of 1:2.5. Cholesterol levels were assayed by a cholesterol auto-analyzer. An HDL sample was diluted (1:10) before injection into the analyzer. Treatments are shown below the peaks. Mean μg cholesterol is shown above the peaks of duplicate samples. Values are mean±S.D. (n=5).

FIG. 1B shows the percent change in cholesterol levels of fresh plasma (control) containing active LCAT and CETP following incubation of plasma with ICP (plaque) or RBC with and without prior R-HDL supplementation (2 mg R-HDL PL/ml plasma). Experimental details are given in the Methods section. Values are mean±S.D. (n=5). Bars with different letters are significantly different, P<0.05 (repeated-measures ANOVA with Tukeys post hoc test).

FIG. 1C shows densitometric scans of TLC plates showing the PC to SPM ratio of 1) control ICP (PC/SPM=0.55), 2) R-HDL-treated ICP (PC/SPM=4.16), 3) HDL-treated ICP (lane 3, PC/SPM=0.65), 4) control R-HDL, 5) ICP-treated R-HDL (PC/SPM=50), 6) control HDL (PC/SPM=2.3) and 7) ICP-treated HDL (PC/SPM=2.0). Following incubation of ICP with TBS or TBS containing R-HDL or HDL at a ICP FC to R-HDL PC or HDL PC ratio of 1:10, undisrupted ICP were pelleted and washed 2× with TBS before lipid extraction.

FIG. 2A shows the amount of cholesterol released from ICP (top) and ICP PL content (bottom) following incubation of ICP with TBS or TBS containing an equal amount of PL from R-HDL made from DMPC or egg PC (apo HDL to PC ratio of 1:4) or from unilamellar DMPC liposomes. The mass ratios of ICP FC to PC on R-HDL and liposomes were 1:10. Values represent means±S.D. of triplicates. Bars with different letters are significantly different, P<0.05 (repeated-measures ANOVA with Tukeys post hoc test).

FIG. 2B shows the amount of cholesterol released from ICP (top) and ICP PL content (bottom) following incubation of ICP with TBS containing an equal amount of PL from R-HDL made from DMPC at apo HDL to DMPC ratio of 1:8, 1:4 or 1:2. The mass ratios of ICP FC to PC on R-HDL and liposomes were 1:10. Values represent means±S.D. of triplicates. Bars with different letters are significantly different, P<0.05 (repeated-measures ANOVA with Tukeys post hoc test).

FIG. 3A shows the effect of incubating ICP with increasing amount of R-HDL on the extent of the release of ICP cholesterol (top) and the change in ICP PL content (middle) and ICP FC to PL ratios (bottom),

FIG. 3B shows the effect of pre-incubating ICP with increasing amount of R-HDL on the extent of release by apo HDL of ICP cholesterol (top) and PL (bottom). ICP were incubated with TBS (control) or R-HDL (at the indicated ICP FC to R-HDL PL ratios) followed by incubation with an equal amount of apo HDL. The levels of cholesterol and PL released from insoluble ICP were measured. The level of apo HDL added to ICP was equal to the PL content of ICP obtained after treatment with R-HDL at a R-HDL PC to ICP FC ratio of 40:1. Values represent mean±S.D. of quadruplicates. Bars with different letters on panels A and B are significantly different, P<0.05 (repeated-measures ANOVA with Tukeys post hoc test).

FIG. 3C shows the lipoprotein cholesterol profiles of hypertriglyceridemic plasma after incubation without (profile I) and with control ICP (profile II), PC-enriched ICP (profile III), or RBC (profile IV), as per the Methods section. ICP were PC-enriched by incubation with R-HDL at a ICP FC to R-HDL PL ratio of 1:40. Cholesterol levels in mg/dl are shown at tops of VLDL, LDL, and HDL peaks.

FIG. 4A shows the morphology of cholesterol crystals in TBS-treated ICP (control) as determined by polarizing microscopy.

FIG. 4B shows the morphology of cholesterol crystals in apo HDL-treated ICP (ICP FC to apo HDL ratio of 1:10) as determined by polarizing microscopy.

FIG. 4C shows the morphology of cholesterol crystals in R-HDL-treated ICP (ICP FC to R-HDL PC ratio of 1:40) as determined by polarizing microscopy.

FIG. 4D shows the morphology of cholesterol crystals in ICP treated initially with R-HDL (as in FIG. 4C) followed by treatment with apo HDL (ICP PL to apo HDL ratio of 1:1) as determined by polarizing microscopy.

FIG. 5A shows a comparison of the effect of TBS (control), native HDL (HDL), PC-enriched HDL and R-HDL on the PL content of treated ICP. Following incubation of ICP with TBS and TBS containing control HDL, PC-enriched HDL or R-HDL (ICP FC to PL on HDL, PL-rich HDL or R-HDL ratio of 1:20), ICP PL content were determined as described herein. PC-enriched HDL was obtained after solubilizing the maximum amount of multilamellar DMPC by freshly isolated HDL. Protein to PL ratios of HDL, DMPC-enriched HDL and R-HDL used in this experiment were 1:0.7, 1:2.5, and 1:6.2, respectively. Values represent mean±S.D. of triplicates. Bars with different letters are significantly different, P<0.05 (repeated-measures ANOVA with Tukey's post hoc test).

FIG. 5B shows a comparison of the effect of TBS (control), native HDL (HDL), PC-enriched HDL and R-HDL on the levels of cholesterol released from ICP on further treatment with apo HDL. Incubations were carried out and PC-enriched HDL were prepared as described in FIG. 5A. Protein to PL ratios of HDL, DMPC-enriched HDL and R-HDL used in this experiment were 1:0.7, 1:2.5, and 1:6.2, respectively. Values represent mean±S.D. of triplicates. Bars with different letters are significantly different, P<0.05 (repeated-measures ANOVA with Tukey's post hoc test).

FIG. 5C shows a comparison of the effect of TBS (control), native HDL (HDL), PC-enriched HDL and R-HDL on the percent change in number of cholesterol crystals relative to control (TBS-treated) ICP. Incubations were carried out and PC-enriched HDL were prepared as described in FIG. 5A. Protein to PL ratios of HDL, DMPC-enriched HDL and R-HDL used in this experiment were 1:0.7, 1:2.5, and 1:6.2, respectively. The percent change in the number of cholesterol crystals relative to control (TBS-treated) ICP (FIG. 5C) was determined after counting the number of cholesterol crystals on 10 microscopic fields of each ICP by using a colony counting computer program. Values represent mean±S.D obtained from 10 microscopic images. Bars with different letters are significantly different, P<0.05 (repeated-measures ANOVA with Tukey's post hoc test).

FIG. 6A shows the density profiles of the cholesterol solubilized from control ICP after its incubation with apo HDL (ICP FC:apo HDL ratio=1:10). Following incubation of insoluble control ICP with apo HDL and subsequent centrifugal pelleting of insoluble ICP, the density profiles of solubilized cholesterol was determined in the upper supernatant fraction via the continuous flow lipoprotein autoprofiler method as described. Arrows indicate the peak banding position of control ICP. Peaks of HDL, LDL and VLDL start to appear on the chart recorder after retention periods of 20-60 seconds, 80-110 seconds and 160-190 seconds, respectively.

FIG. 6B shows the density profiles of the cholesterol solubilized from control ICP after its incubation with R-HDL (ICP FC:R-HDL PL ratio=1:40). Density profiles of solubilized cholesterol were determined as in FIG. 6A.

FIG. 6C shows the density profiles of the cholesterol solubilized from control ICP after its incubation with R-HDL-treated ICP after further incubation with apo HDL ICP PL:apo HDL ratio=1:1). ICP PL:apo HDL ratio=1:1). Density profiles of solubilized cholesterol were determined as in FIG. 6A.

FIG. 6D shows the distribution among density gradient fractions of phospholipid and apo A-I moieties of control R-HDL. Following density gradient ultracentrifugation of R-HDL, and subsequent fractionation of density gradient samples into 22 fractions, the distribution of PL and apo A-I among density gradient fractions were determined by assaying the level of apo A-I and PL in each gradient fraction.

FIG. 6E shows the distribution among density gradient fractions of phospholipid and apo A-I moieties of control R-HDL (R-HDL PL:ICP FC ratio=40:1) incubated with insoluble ICP. Apo A-I and PL were assayed as described in FIG. 6D.

FIG. 7A show the effect of hydrolyzing SPM on ICP by sphingomyelinase on the ICP SPM to PC ratio. Following incubation of ICP in 0.25 M Tris buffer containing 20 mM MgCl₂ with 50 units of bacterial sphingomyelinase for 6 hrs at 37° C., the reaction of sphingomyelinase was stopped by adding EDTA and a subsequent wash. A change in the SPM to PC ratio of insoluble ICP was examined by TLC. SPM to PC ratios of control ICP (top) and sphingomyelinase-treated ICP (bottom) were 1.69 and 0.49, respectively.

FIG. 7B show the effect of hydrolyzing SPM on ICP by sphingomyelinase on the release of ICP cholesterol by apo HDL or R-HDL. ICP sphingomyelinase treatment was as described in FIG. 7A. Release of cholesterol from control or sphingomyelinase-treated ICP was then measured after incubation of ICP at ICP FC to apo HDL ratio=1:2.5 or ICP FC to R-HDL PC ratio of 1:10. Values represent mean±S.D. of triplicates. * indicates values are significantly different at P<0.05 (paired t-test).

FIG. 7C the effect of enriching artificial multilamellar SPM vesicles with FC on the movement of PC between R-HDL and SPM vesicles. Following incubation of R-HDL with FC-rich-multilamellar SPM vesicles (SPM/FC ratio=1, labeled as SPM/FC+) and FC-free multilamellar SPM vesicles (labeled as SPM/FC−) at a SPM to R-HDL PC ratio of 1:5, transfer of PC from R-HDL to undisrupted multilamellar SPM vesicles was examined by TLC method as described in the methods. Residual (undisrupted) vesicles were pelleted by centrifugation and washed with TBS prior to lipid extraction and TLC. Lanes: (1) Control SPM/FC+ vesicles, (2) Undisrupted (pelleted) SPM/FC+ vesicles recovered after incubation with R-HDL, (3) Soluble R-HDL fraction after incubation with SPM/FC− vesicles, (4) Control R-HDL, (5) Control SPM/FC− vesicles, (6) Residual pelleted SPM/FC− vesicles recovered after incubation with R-HDL, and (7) Soluble fraction after incubation of R-HDL with SPM/FC− vesicles, and (8) Control R-HDL.

FIG. 8A shows representative photographs of rabbit aortas from control rabbit after 15 weeks of normal rabbit chow diet.

FIG. 8B shows representative photographs of rabbit aortas from rabbits after 6 weeks of cholesterol-rich atherogenic diets and 9 weeks of normal rabbit chow diet (Group II).

FIG. 8C shows representative photographs of rabbit aortas from rabbits after 6 weeks of cholesterol-rich atherogenic diets, 4 weeks of normal rabbit chow diet and 5 weeks infusion of DMPC while on a chow diet (Group I).

FIG. 9 shows the visualization of plaque regression as determined by non-invasive magnetic resonance imaging (MRI) technique. The top panel shows representative cross sections of the rabbit aorta obtained from the control cholesterol-fed rabbits (Group II), while the bottom panels shows cross sections of rabbit aorta from the DMPC infused cholesterol-fed rabbit (Group I).

### DETAILED DESCRIPTION

Definitions The terms “prevention”, “prevent”, “preventing”, “suppression”, “suppress” and “suppressing” as used herein refer to a course of action (such as administering a compound or pharmaceutical composition) initiated prior to the diagnosis or onset of a clinical symptom of a disease state or condition so as to prevent or reduce the occurrence of the disease state or condition. Such preventing and suppressing need not be absolute to be useful.

The terms “treatment”, “treat” and “treating” as used herein refers a course of action (such as administering a compound or pharmaceutical composition) initiated after the diagnosis or onset of a clinical symptom of a disease state or condition so as to eliminate or reduce the occurrence of the disease state or condition. Such treating need not be absolute to be useful.

The term “in need of treatment” as used herein refers to a judgment made by a caregiver that a patient requires or will benefit from treatment. This judgment is made based on a variety of factors that are in the realm of a caregiver's expertise, but that includes the knowledge that the patient is ill, or will be ill, as the result of a disease state or condition that is treatable by a method or compound of the disclosure.

The term “in need of prevention” as used herein refers to a judgment made by a caregiver that a patient requires or will benefit from prevention. This judgment is made based on a variety of factors that are in the realm of a caregiver's expertise, but that includes the knowledge that the patient will be ill or may become ill, as the result of a disease state or condition that is preventable by a method or compound of the disclosure.

The term “individual”, “subject” or “patient” as used herein refers to any animal, including mammals, such as mice, rats, other rodents, rabbits, dogs, cats, swine, cattle, sheep, horses, or primates, and humans. The term may specify male or female or both, or exclude male or female; and

The term “therapeutically effective amount” as used herein refers to an amount of a compound either alone or contained in the pharmaceutical composition that is capable of having any detectable, positive effect on any symptom, aspect, or characteristics of a disease state or condition. Such effect need not be absolute to be beneficial and may be made in relation to treatment or prevention.

In one embodiment, the teachings of the present disclosure provide for the treatment of disease states and conditions related to coronary artery disease (CAD), such as, but not limited to, atherosclerosis, in a subject in need of such treatment. The method of treatment comprises the steps of identifying a subject in need of such treatment and administering a therapeutically effective amount of a composition of the present disclosure, or pharmaceutical composition containing such composition. In one embodiment, the composition is a lipid composition comprising at least one phospholipid and, optionally, at least one protein. In a specific embodiment, the phospholipid is a phosphatidylcholine species and the protein is a apolipoprotein. In a further specific embodiment, the composition in PC or R-HDL. The described composition may have a number of effects on the subject. In one embodiment, the composition is capable of enhancing the removal, transport or dissolution of cholesterol from atherosclerotic plaques; in an alternate embodiment, the composition is capable of reducing the volume of the atherosclerotic plaque; in yet another alternate embodiment, the composition is capable of increasing the stability of the atherosclerotic plaque decreasing the change of plaque rupture. The composition may exert one or more than one of the effects described, or may exert other effects. Such effects may be direct or indirect or aided by other physiological processes in the subject in one embodiment, the described composition may enhance the removal, transport or dissolution of cholesterol from plaques by allowing the native HDL of the subject to more efficiently receive the cholesterol from the plaque. Such effects are a positive factor in treating CAD and diseases states and conditions related to CAD. Any of the above effects may be accompanied by changes in the composition of the atherosclerotic lesions.

In an alternate embodiment the teachings of the present disclosure provide for the prevention of disease states and conditions related to coronary artery disease (CAD), such as, but not limited to, atherosclerosis, in a subject in need of such prevention. The method of prevention comprises the steps of identifying a subject in need of such prevention and administering a therapeutically effective amount of a composition of the present disclosure, or pharmaceutical composition containing such composition. In one embodiment, the composition is a lipid composition comprising at least one phospholipid and, optionally, at least one protein. In a specific embodiment, the phospholipid is a phosphatidylcholine species and the protein is a apolipoprotein. In a further specific embodiment, the composition in PC or R-HDL. The described composition may have a number of effects on the subject. In one embodiment, the composition is capable of enhancing the removal, transport or dissolution of cholesterol from atherosclerotic plaques; in an alternate embodiment, the composition is capable of reducing the volume of the atherosclerotic plaque; in yet another alternate embodiment, the composition is capable of increasing the stability of the atherosclerotic plaque decreasing the change of plaque rupture. The composition may exert one or more than one of the effects described, or may exert other effects. Such effects may be direct or indirect or aided by other physiological processes in the subject; in one embodiment, the described composition may enhance the removal, transport or dissolution of cholesterol from plaques by allowing the native HDL of the subject to more efficiently receive the cholesterol from the plaque. Such effects are a positive factor in preventing CAD and diseases states and conditions related to CAD. Any of the above effects may be accompanied by changes in the composition of the atherosclerotic lesions.

The methods of the treating and preventing discussed herein may also comprise further administering of one or more additional therapeutic agents agent in combination with those compounds or pharmaceutical compositions.

The described compositions may be administered to the subject as is known in the art and/or determined by a healthcare provider. Certain modes of administration are provided herein and should not be considered as limiting examples. Furthermore, the described compositions may be administered with other agents. Such other agents may be agents that increase the activity of the described compositions, such as by limiting their degradation or inactivation or by increasing their absorption or activity. The described compositions may further comprise pharmaceutically acceptable carriers including, but not limited to, vehicles, adjuvants, excipients, or diluents. Typically, the pharmaceutically acceptable carrier is chemically inert to the active compounds and has no detrimental side effects or toxicity under the conditions of use. The pharmaceutically acceptable carriers can include polymers and polymer matrices. Examples of such carriers can be found in “Remington, The Science and Practice of Pharmacy, 20^{th} Edition, Lippincott, Williams & Wilkins, Baltimore, Md.).

The described compositions (whether alone or in combination with other therapeutic agents) may be formulated for oral administration or parenteral administration or other forms of administration as known in the art. Oral administration includes, but is not limited to, formulations of liposomes, emulsion and particles, such as HDL particles. Parenteral administration includes, but is not limited to, intravenous administration, subcutaneous administration, intramuscular administration, intradermal administration, intrathecal administration, intraarticular administration, intracardiac administration, retrobulbar administration and administration via implants, such as sustained release implants. The composition can also be administered intranasally (nose drops) or by inhalation via the pulmonary system, such as by propellant based metered dose inhalers or dry powders inhalation devices. Other dosage forms are potentially possible such as administration transdermally, via patch mechanism or ointment. In one embodiment, PC or R-HDL are administered by intravenous infusion.

The described composition is administered in therapeutically effective amount. The therapeutically effective amount will, of course, vary depending upon known factors, such as the pharmacodynamic characteristics of the particular composition and its mode and route of administration; the age, health and weight of the subject; the severity and stage of the disease state or condition; the kind of concurrent treatment; the frequency of treatment; and the effect desired. The total amount of the described composition to be administered will also be determined by the route, timing and frequency of administration as well as the existence, nature, and extent of any adverse side effects that might accompany the administration and the desired physiological effect. It will be appreciated by one skilled in the art that various conditions or disease states, in particular chronic conditions or disease states, may require prolonged treatment involving multiple administrations of the described composition.

The compounds and pharmaceutical compositions described in the instant disclosure can be administered by any conventional method available for use in conjunction with compounds or pharmaceutical compositions, either alone or in combination with additional therapeutic agents.

Dosage forms of the composition described herein (forms suitable for administration) contain from about 0.1 mg to about 500 mg of active ingredient (i.e. the compounds disclosed) per unit. In these compositions, the active ingredient will ordinarily be present in an amount of about 0.5-95% weight based on the total weight of the composition. Multiple dosage forms may be administered as part of a single treatment.

One skilled in the art will appreciate that suitable methods of administering a composition of the present invention to a subject are available, and, although more than one route can be used to administer a particular composition, a particular route can provide a more immediate and more effective reaction than another route.

### EXAMPLES

### ICP Composition

Cholesterol (comprising free cholesterol, FC, and cholesterylester, CE) was the major lipid constituent of insoluble ICP material used in the present disclosure, comprising 66.5±12.6% of the total mass. Phospholipids (PL), proteins, and triglycerides comprised 15.7±8.9%, 17.4±10.9% and 1.4±1.1% of the total mass, respectively. FC to PL ratios ranged from 0.8 to 3.1 (mean, 2.1±0.5), FC to CE ratios ranged from 0.6 to 4.8 (mean, 1.9±1.2), and SPM to PC ratios ranged from 1.2 to 4.2 (mean, 1.8±0.8).

### Effect of Incubating ICP with HDL, apo HDL, R-HDL or PC Liposomes on the Release of ICP Cholesterol and Alteration in ICP Lipids

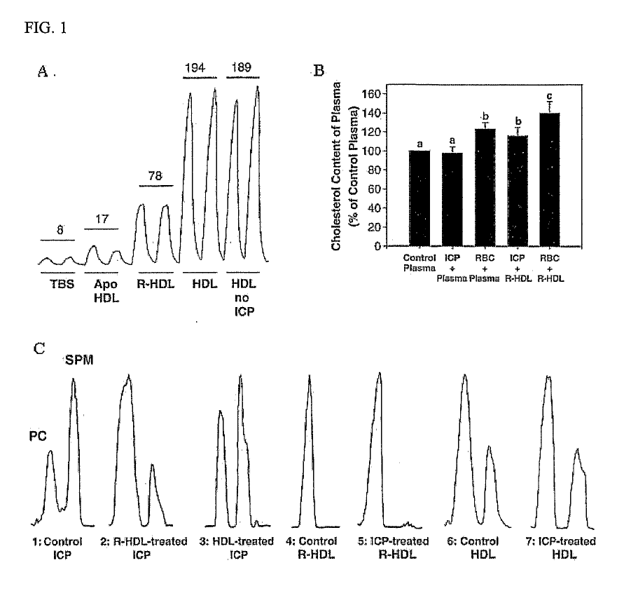

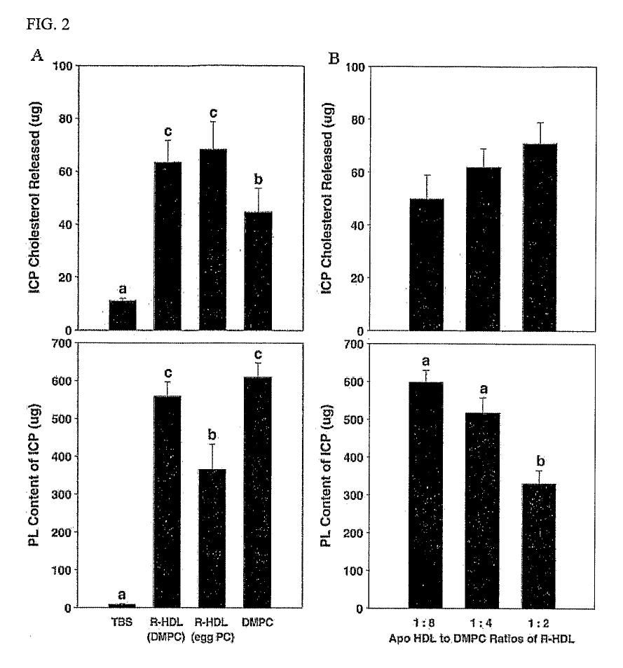

FIG. 1A shows the relative levels of cholesterol released from ICP following incubation with apo HDL, R-HDL, or HDL (each delivered in TBS vehicle). Little cholesterol was released by TBS alone, and slightly more was released by TBS containing apo HDL. However, R-HDL released markedly greater (4.5-10 fold) quantities of cholesterol (largely FC, data not shown) from ICP than by TBS or apo HDL. No cholesterol was released from ICP by HDL, since HDL cholesterol content was unchanged by incubation with ICP (FIG. 1A). We previously reported that fresh plasma containing active cholesterol acyltransferase (LCAT) and cholesterylester transfer proteins (CETP) promotes cholesterol release from autologous RBC (32). Accordingly, fresh plasma released significant amounts of cholesterol from RBC, but did not release cholesterol from ICP (FIG. 1B). R-HDL-supplemented fresh plasma released more cholesterol from RBC and enabled the release of ICP cholesterol (FIG. 1B). These results indicate that ICP cholesterol is releasable but not as readily as that on normal cell membranes.

Incubating ICP with R-HDL released only small portion (<15%) of FC on ICP, but the ICP FC/PL ratio was markedly lowered from 2.5 to 0.48 (data not shown), suggesting that ICP PL content was increased by this treatment. TLC analysis (FIG. 1C) showed that PC/SPM ratio of control ICP increased from 0.55 to 4.16 following its incubation with R-HDL (scans 1 and 2) without a noticeable transfer of SPM from ICP to R-HDL (scans 4 and 5). Together, these results indicate that the major effect of R-HDL on ICP lipid composition is increased PL, which, in turn, is due to the acceptance of PC from R-HDL. Incubating ICP with HDL resulted in a small increase in the PC/SPM ratio of ICP (FIG. 1C, scans 1 and 3) and a small decrease of PC/SPM ratio of HDL (FIG. 1C, scans 6 and 7), indicating that ICP accepted some PC from HDL even though little or no cholesterol was released (FIG. 1C).

FIG. 2A shows the effect of incubating ICP with an equal amount of DMPC from liposomes or R-HDL on the levels of cholesterol released from ICP and the concentration of PL concentration in ICP. Liposomes were significantly less effective than R-HDL in releasing cholesterol from ICP but somewhat more effective in enriching ICP with PL (FIG. 2A-top and bottom). These data indicate that the uptake of R-HDL PC by ICP does not require apolipoproteins as the DMPC liposomes contained no apolipoproteins. Further study showed that R-HDL prepared from egg PC tends to release more cholesterol from ICP but was significantly less effective in enriching ICP with PC than R-HDL prepared from DMPC (FIG. 2A-top and bottom). The effect of the change in PL (in this case DMPC) to protein ratio of R-HDL on the release of cholesterol from ICP and the increase of PL content of ICP is shown in FIG. 2B. R-HDL that was more enriched with PL (ratios of 1:2, 1:4 and 1:8 apo AI to DMPC) released less cholesterol from ICP but transferred more PC from R-HDL to ICP (FIG. 2B-top and bottom). Further, incorporation of FC into R-HDL (DMPC:FC=10:1) significantly lowered the ability of R-HDL to increase PL content of ICP (data not shown).

### Effect of Incubating ICP with Increasing Levels of R-HDL on the Release of ICP Cholesterol, Change in ICP PL, and on the Ability of apo HDL to Release Cholesterol from ICP

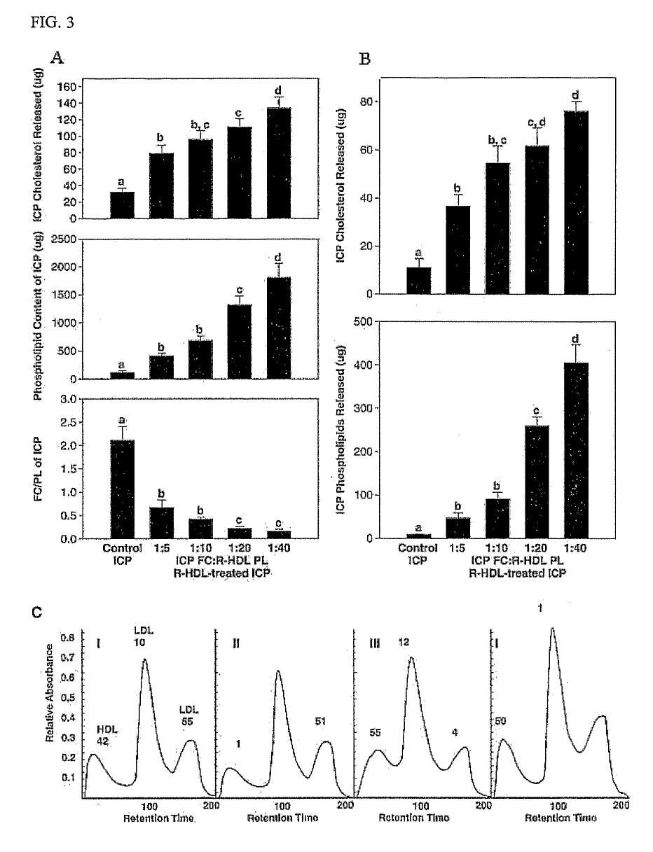

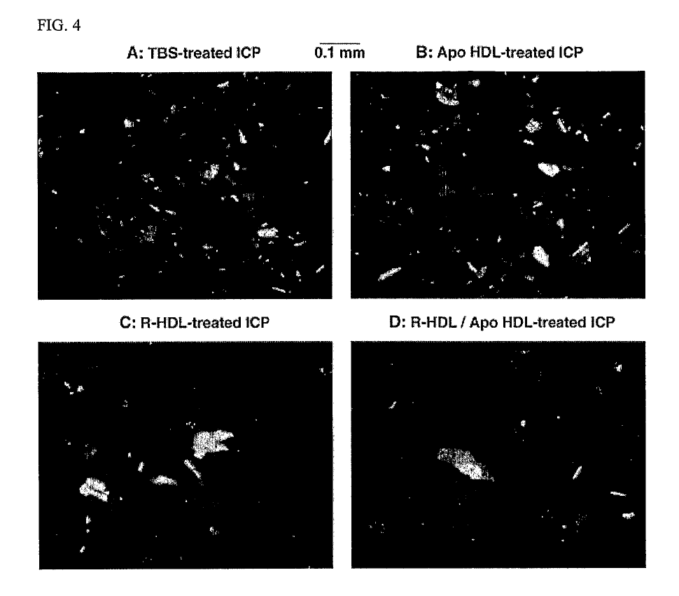

Cholesterol and/or PL levels were determined in the supernatant fraction and in insoluble ICP pellets after incubation of ICP with R-HDL at ICP FC to R-HDL PL ratios of 1:0, 1:5, 1:10, 1:20, and 1:40 (FIG. 3A-C). FIG. 3A shows that increases in levels of R-HDL as compared to plaque FC in the incubation mixtures increased the levels of cholesterol released from ICP (top) and ICP PL contents (middle), and decreased the ICP FC/PL ratios (bottom) in a dose-dependent manner. Similarly, DMPC liposomes caused a similar dose-dependent increase in the release of cholesterol from ICP, as well as likewise enrichment of ICP with PL (data not shown). At all ratios, the amount of PC transferred from R-HDL to ICP were much more than the amount of cholesterol released from ICP into R-HDL (FIG. 3A, top and middle), thereby decreasing ICP FC/PL ratios (FIG. 3A, bottom). Although apo HDL has little inherent ability to release cholesterol from control ICP (FIG. 1A), the R-HDL-mediated increase in ICP PL content enabled apo HDL to release ICP cholesterol and PL in a subsequent incubation (FIG. 3B). The levels of ICP cholesterol and PL released by an equal amount of apo HDL increased proportionately with the increase in ICP PL following incubation with increasing levels of R-HDL (FIGS. 3A and 3B). The levels of PL released from PC-enriched ICP by apo HDL were 1.2-5× greater than the levels of released cholesterol (FIG. 3B, top and bottom). Further, under these conditions, apo HDL released cholesterol and PL simultaneously and rapidly, with almost all (90%) of the release occurring within 2 hr after incubation (data not shown). Incubation of PL-enriched ICP with apo HDL solubilized only 50-65% of PL on ICP. Further, additional cholesterol and PL were released from apo HDL-treated ICP upon addition of fresh apo HDL for a second time (data not shown). Approximately 20-30% of FC on ICP materials was observed to be released after treatment with R-HDL followed by two treatments with apo HDL (data not shown).

Further studies revealed that the R-HDL-mediated increase in PC content of ICP enabled fresh plasma to release cholesterol from ICP, resulting in 10.5±3.4% (n=3) higher cholesterol in plasma cholesterol after incubation with PL-enriched ICP. FIG. 3C compares the cholesterol profiles of plasma samples that were incubated with control ICP (profile II), PC-enriched ICP (profile III), or RBC (profile IV); profile 1 is a control. Plasma cholesterol decreased by 11% after incubation with ICP by a decrease in LDL and HDL cholesterol (compare profiles I and II) but increased by 13% after incubation with PC-enriched ICP by an increase in HDL cholesterol (compare profiles I and III). In contrast, following incubation with RBC, total plasma cholesterol increased 31% owing to an increase in cholesterol content of all lipoprotein fractions (FIG. 3C, profiles I and IV). These data indicate that plasma releases cholesterol from ICP less readily than from normal cell membranes.

### Effect of Treatment of ICP with R-HDL, apo HDL or Control and PC-enriched HDL on ICP Cholesterol Monohydrate Crystals

FIGS. 4A-D shows the effect of incubating ICP with R-HDL and apo HDL on ICP cholesterol monohydrate crystals, as revealed by polarizing microscopy. Control (TBS-treated) ICP contained cholesterol crystals in rhomboid plates and needle-shaped particles in many sizes (FIG. 4A). The number and shape of crystals were unaffected by incubation with apo HDL (FIG. 4B). However, the number of cholesterol crystals decreased by 84% (FIG. 4C) after treatment with R-HDL (ICP FC/R-HDL PL ratio of 1:40) and decreased by a total of 91% after further incubation with apo HDL (FIG. 4D). Some large crystals remained, however (FIG. 4D, center).

### Effect of In Vitro Enrichment of HDL with PC on the Potencies of HDL to Alter the ICP PL Level and Cholesterol Crystal Number and to Influence the Release of ICP Cholesterol by apo HDL

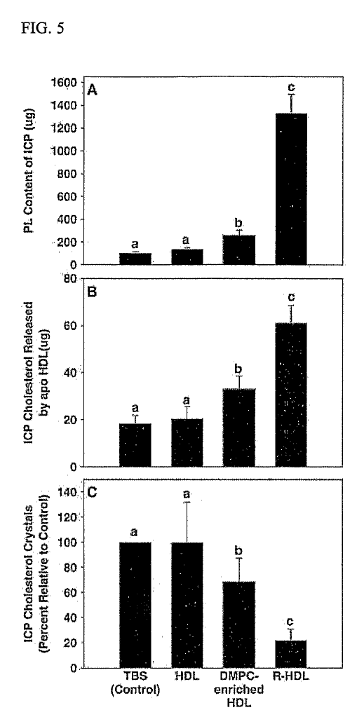

FIGS. 5A-C show the effect of enriching HDL with PC in vitro on the ability of HDL to increase ICP PL content (FIG. 5A), to release ICP cholesterol by apo HDL (FIG. 5B), and to change the number of ICP cholesterol crystals (FIG. 5C). Plasma HDL (PL/protein ratio=0.7) had minimal ability to increase ICP PL content over control, but that ability was enhanced significantly by enriching HDL with PC (PL/protein=2.5), (FIG. 5A). While the levels of cholesterol released from ICP by apo HDL were not affected after treatment with control HDL (FIG. 5B), they increased significantly after the ICP were treated with PC-enriched HDL. The number of cholesterol crystals on ICP was not altered following treatment of ICP with control HDL but significantly decreased following treatment with PC-enriched HDL (FIG. 5C). Compared to DMPC-enriched HDL, R-HDL (PL/protein=1:6.2) was 5× more effective in increasing ICP PL content, 3.1× more effective in decreasing the number of cholesterol crystals on ICP, and 1.8× more effective in influencing the release of ICP cholesterol by apo HDL (FIGS. 5A-C).

### Density Profiles and Composition of Soluble Cholesterol-containing Particles Released from ICP by R-HDL and apo HDL

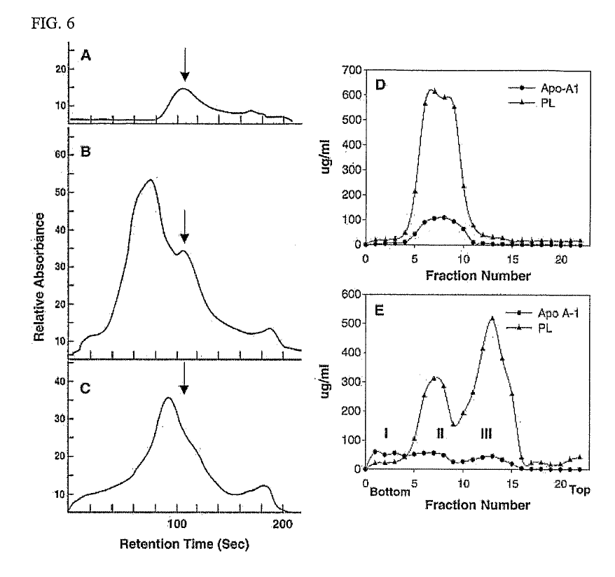

FIGS. 6A-E show density spectrums of soluble cholesterol-containing particles released from ICP by treatment with apo HDL and R-HDL. Little cholesterol was released from ICP by apo HDL and was associated with particles having LDL-like densities (FIG. 6A, arrow). After incubation with R-HDL, cholesterol released from ICP was associated with two major distinct peaks, one in the LDL and the other in the HDL density range (FIG. 6B). It is evident from FIG. 6B that a small amount of cholesterol released from ICP by R-HDL was associated with the VLDL density region (top of density gradient tube) where CE droplets recovered (25). Cholesterol released from PL-enriched ICP after further treatment with apo HDL treatment was associated with a broad peak having a density somewhat greater than that of LDL density (FIG. 6C).

FIGS. 6D and E show further the distribution of apo A-I and PL in control R-HDL (FIG. 6D) and R-HDL after incubation with ICP (FIG. 6E). Apo A-I and PL on control R-HDL were recovered as a single peak in the HDL density region (FIG. 6D). In contrast, apo A-I in the ICP-treated R-HDL was recovered in a PL-poor peak in the bottom of the density gradient tube (FIG. 6E, peak I) and in two peaks of PL-containing particles in the middle density region (FIG. 6E, peaks II and III). The applicants previously reported that apo A-I was associated with the liposome-like vesicles isolated from human atherosclerotic lesions (24). Accordingly, a band corresponding to apo A-I in SDS gels of insoluble ICP material increased upon incubation with R-HDL (data not shown). FIGS. 6B and 6E taken together demonstrate the formation of a free apo A-I peak and two peaks containing apo A-I, PL, and cholesterol after R-HDL interacts with ICP. These data suggests that a new R-HDL-like particle was formed by R-HDL-mediated PC-enrichment of ICP followed by association of ICP PC and cholesterol with apo HDL.

### Role of Enrichment of SPM and FC on ICP in Mediating Cholesterol or PC Transfer

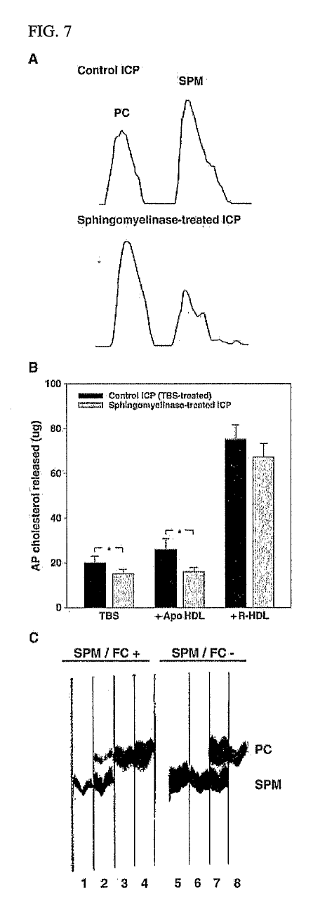

FIGS. 7A and B show that high levels of SPM, which is more tightly associated with cholesterol than other PL components in plasma membranes (33), does not explain the poor release of ICP cholesterol by R-HDL and apo HDL. Treatment of ICP with bacterial sphingomyelinase hydrolyzed 70% of ICP SPM. This treatment decreased the ICP SPM/PC ratio from 1.6 to 0.49 (FIG. 7A) and increased the FC/PL ratio (data not shown), but did not elicit greater ICP cholesterol release by apo HDL, R-HDL (FIG. 7B) or HDL (data not shown). Rather, the release of ICP cholesterol into TBS, apo HDL, or R-HDL was reduced by prior sphingomyelinase treatment of ICP (FIG. 7B). As a model of ICP, artificial multilamellar SPM vesicles enriched with FC (SPM/FC ratio=1) or containing no FC (control SPM) were incubated with R-HDL. Then, PC and SPM content in residual insoluble multilamellar SPM vesicles and solubilized vesicles were determined by TLC (FIG. 7C). After incubation of FC-enriched multilamellar SPM vesicles (SPM/FC+, FIG. 7C, lane 1) with R-HDL (lane 4), insoluble multilamellar vesicles contained not only SPM but also PC (lane 2), while the soluble fraction contained only PC (lane 3). In contrast, following incubation of FC-free multilamellar SPM vesicles (SPM/FC−, lane 5) with R-HDL (lane 8), the residual insoluble multilamellar vesicles contained SPM and no PC (lane 6), and the upper soluble fraction contained both SPM and PC (lane 7). These data (FIG. 7C) suggest that the transfer of PC from R-HDL to SPM vesicles requires high levels of FC within the SPM vesicles. The abilities of FC-rich SPM vesicles and ICP to accept PC from R-HDL are similar, suggesting that the combination of high FC and SPM on ICP may be responsible for these transfers.

### DPMC Treatment Reduces Plasma Total Cholesterol Levels and Atherosclerotic Plaque Volumes in a Rabbit Model of Atherosclerosis

To induce the development of atherosclerosis, 8 weeks old male New Zealand white rabbits weighing 4-5 lbs (n=24) were fed with cholesterol-rich atherogenic diets (a diet containing 2% cholesterol and 6% peanut oil) for 6 weeks. During this atherogenic diet period, 11 animals died due to liver failure. The survived cholesterol-fed rabbits (n=13) were then fed with normal rabbit chow for 4 weeks. A second group of rabbits were maintained on a normal rabbit chow diet for the 10 week period. At 10 weeks, the cholesterol-fed rabbits were divided into two groups each: (i) a treatment group (group I; n=8); and (ii) and a no treatment group (group II; n=5). The group I rabbits received weekly intravenous infusions (5 ml) of unilamellar DMPC liposome (300 mg/kg) in normal saline for 5 weeks. The group II animals (control) received 5 ml normal saline. During this 5-week period both group I and group II animal received normal rabbit chow; in addition the second (control) group rabbits were also maintained on normal rabbit chow for an additional 5 weeks.

Plasma total cholesterol levels of rabbits were determined as follows; after 15 weeks of normal rabbit chow (control rabbits): 40±6.1 mg/dl; after 6 weeks of cholesterol-rich atherogenic diets: 2,918±489 mg/dl; after 6 weeks of cholesterol-rich atherogenic diets—4 weeks of normal rabbit chow diet and 5 week infusion of normal saline while on a chow diet: 972±442 mg/dl; and after 6 week cholesterol-rich diet—4 weeks normal rabbit chow and 5 weeks DMPC infusion while on a chow diet: 1159±690 mg/dl.

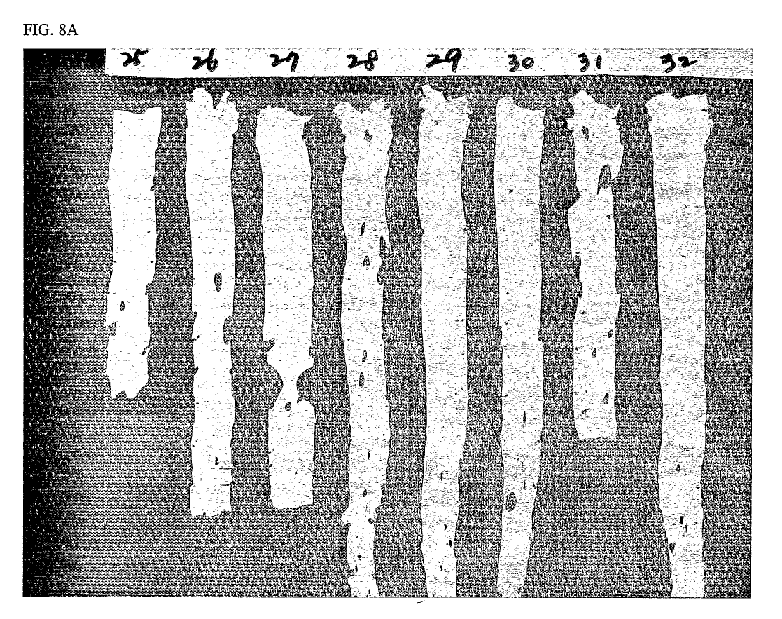

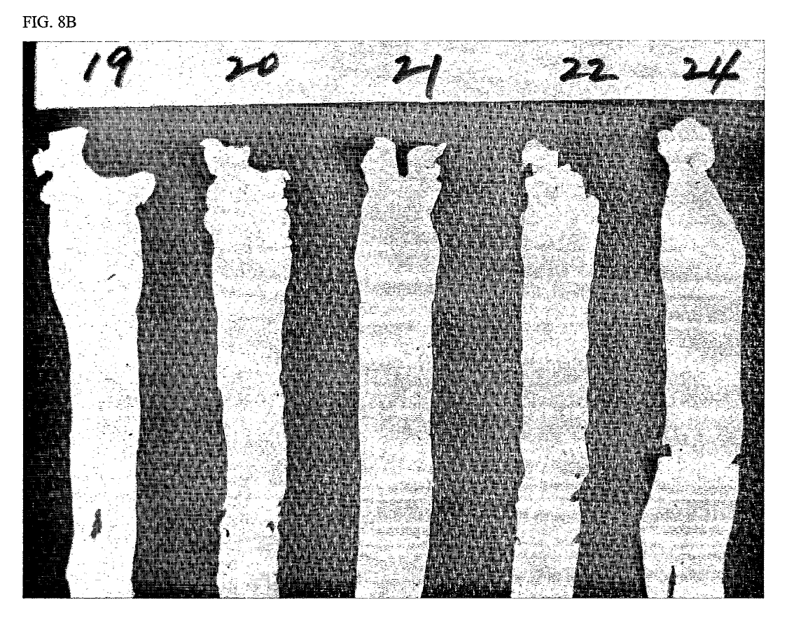

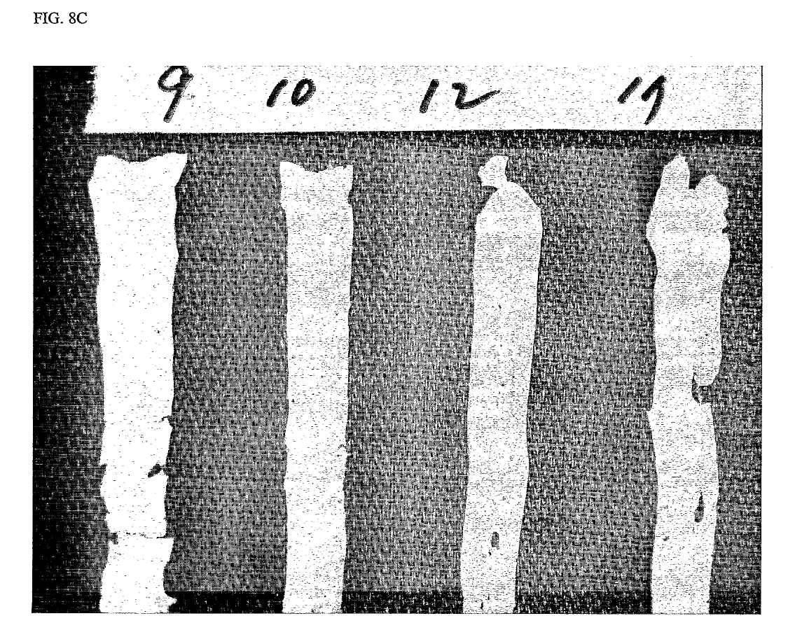

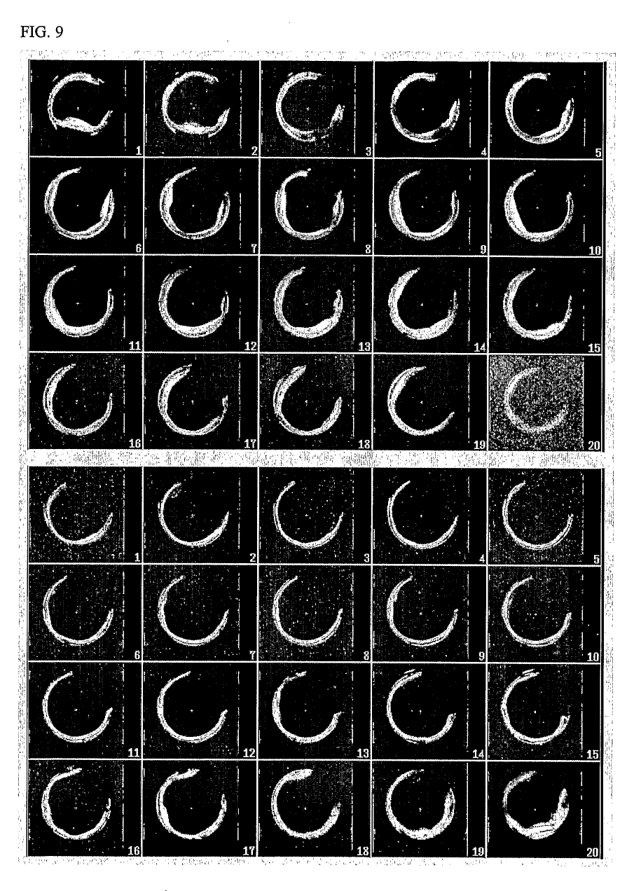

Normal rabbit chow-fed control rabbits and rabbits fed the cholesterol-rich atherogenic diets (both with infusion of DPMC and without) were sacrificed and the degree of atherosclerosis in the rabbit aorta were examined by a magnetic resonance imaging method. Initial analysis of the results indicates a 20-70% reduction of atherosclerotic plaque volumes after treatment with DPMC as compared to treatment with saline alone. FIGS. 8A-C show photographs of rabbit aortas from control rabbit (after 15 weeks of normal rabbit chow diet) (FIG. 8A), after 6 weeks of cholesterol-rich atherogenic diets and 9 weeks of normal rabbit chow diet (FIG. 8B) and after 6 weeks of cholesterol-rich atherogenic diets, 4 weeks of normal rabbit chow diet and 5 weeks infusion of DMPC while on a chow diet (FIG. 8C). FIG. 9 shows the visualization of plaque regression as examined by non-invasive magnetic resonance imaging (MRI) technique. FIG. 9 shows 20 cross sections of the rabbit aorta obtained from the control cholesterol-fed rabbits (Group II, top panel) and DMPC infused cholesterol-fed rabbit (Group I, bottom panel). Areas of thickness around the cross section of the aorta represent areas of plaque formation. Comparing the top and bottom panels, it is clear that DMPC administration caused a significant regression of atherosclerotic lesions. FIGS. 8B-C and FIG. 9 showed that DMPC treatment significantly reduces plaque volume (formation) on the rabbit aortas examined.

A human study examining the effect of the infusion of recombinant HDL (complexes apo A-I milano and phosphatidylcholine) on the regression of atherosclerotic lesions has been reported (50); however, the results showed only a 4.2% regression of atherosclerotic lesion, which is much lower than that presented in the present disclosure. Furthermore, this study showed that apo A-I milano is the active component responsible for the reported regression of atherosclertoic lesions. In contrast, the present disclosure demonstrates that the active component is the phospholipids, such as DPMC.

### Discussion

Isotopic studies in vitro and in vivo indicate that FC constantly moves among lipoproteins and between cells and lipoproteins through a nonspecific aqueous diffusion process (5, 6, 34). Cholesterol diffuses down a potential chemical gradient and partitions into PL-rich particles (5, 6). The direction of net FC transfer is determined by FC to PL ratios of the donor and acceptor particles in this exchange (5, 6). Thus, the relative FC to PL ratios of lipoproteins and cell membranes will determine whether lipoproteins will be donors or acceptors of cellular cholesterol. Whole serum, HDL, and intact erythrocytes effectively remove cholesterol from cultured cholesterol-loaded macrophages (35). The present disclosure is the first to compare directly the HDL-mediated removal of cholesterol from normal cell membranes and ICP prepared from human atherosclerotic lesions. The ICP preparation used herein may contain PL bilayers, cholesterol monohydrate crystals, large extracellular liposome-like vesicles, and possibly intact foam cells and their debris containing cholesterylester droplets, since these components are recoverable by others (15, 25) within the same density regions (d=1.02-1.07 g/ml) as the ICP preparations used herein.

It is generally believed that SR-B1 receptors and the ABCA1 transporter play an important role in removing cholesterol by HDL and apo A-I from atherosclerotic lesions in vivo (7-9). However, a recent study indicated that neither the ABCA1 transporter or SR-B1 receptors is required for cholesterol efflux in vascular endothelial cells (36), suggesting that aqueous diffusion is the major process for removing cholesterol from vascular endothelium. The present disclosure demonstrates that ICP cholesterol was not released by fresh plasma, HDL and apo HDL. Only R-HDL or PC liposomes released ICP cholesterol in a dose-dependent manner likely via the process of aqueous diffusion. However, the release of cholesterol from ICP accompanied the transfer of 8-17× more PC from R-HDL or liposomes to ICP. The R-HDL-mediated enrichment of ICP with PC allowed the release of ICP cholesterol by apolipoproteins and fresh plasma. Remarkably, the transfer of PC from R-HDL or PC liposomes to ICP decreased the number of cholesterol crystals. It has been reported that the intravenous administration of aqueous dispersions of PL were shown to cause rapid, substantial shrinkage of lipid-rich arterial lesions in animals, where the antiatherogenic effects of PL particles may result from their ability to act as synthetic mediators of RCT from peripheral tissues to the liver (37, 38). The present disclosure shows that PL may play a role in regressing atherosclerotic lesions that contain cholesterol crystals in vivo by remodeling plaques via enrichment with PC, allowing cholesterol, which otherwise remains resistant to release by native HDL or apolipoprotein, to be released.

The foregoing description illustrates and describes the compounds and methods of the present disclosure. Additionally, the disclosure shows and describes only certain embodiments of the compounds and methods but, as mentioned above, it is to be understood that the teachings of the present disclosure are capable of use in various other combinations, modifications, and environments and is capable of changes or modifications within the scope of the inventive concept as expressed herein, commensurate with the above teachings and/or the skill or knowledge of the relevant art. The embodiments described hereinabove are further intended to explain best modes known of practicing the invention and to enable others skilled in the art to utilize the invention in such, or other, embodiments and with the various modifications required by the particular applications or uses of the invention. Accordingly, the description is not intended to limit the invention to the form disclosed herein. All references cited herein are incorporated by reference as if fully set forth in this disclosure.

### METHODS

### Reagents

Egg PC, egg SPM, dimiristoylphosphatidylcholine (DMPC), and FC were purchased from Avanti Polar Lipid Co., Birmingham, Ala. Bacterial (Bacilus cereus) sphingomyelinase was purchased from Sigma Chemical Co., St. Louis, Mo. Enzymatic assay reagents for cholesterol, FC and PL and an immunoassay kit for apo A-I were purchased from Wako Diagnostic Co., Richmond, Va.

### Preparation of ICP

Human atherosclerotic aortas (8 to 30 hours post-mortem), evaluated by a pathologist, were obtained from the Tissue Procurement Facility at the University of Alabama Medical Center. The Institutional Review Board at the University of Alabama approved the use of human tissues in this study. Tissues were rinsed 5 times with Tris-buffered saline (TBS) (0.15 M NaCl-0.01 M Tris, pH 7.4) containing 0.1% EDTA, 20 μM butylated hydroxytoluene, and proteinase inhibitor cocktails. Aortic intima containing grossly visible plaques (i.e. yellow-white to golden yellow streaks and/or patches) was stripped from the media, washed 4× with TBS, and minced finely with scissors. After large tissue fragments were pelleted by a low speed centrifugation (700× g for 10 min.), they were gently homogenized, and the homogenates were centrifuged again. ICP in the pooled upper supernatant fractions were fractionated by a modification of a density gradient ultracentrifugation method described previously (25). Briefly, the density of the supernatant was adjusted to d=1.21 g/ml and placed at the bottom of ultracentrifuge tubes (Beckman SW 41 rotor tubes) containing 4 ml TBS layered on top of 4 ml KBr solution (d=1.12 g/ml) (26). After centrifugation at 40,000 rpm for 3 hrs, flocculent lipid particles banded at d=1.02 g/ml to d=1.07 g/ml density ranges (lower- to upper-middle portion) of the density gradient tube. Greater than 65% of the cholesterol in the density gradient tube was in the d=1.02-1.07 g/ml density fraction. Following removal of KBr from pooled middle fractions by dialysis against TBS at 4° C. for 16 hr and subsequent centrifugation at 10,000 rpm for 15 min. in a microcentrifuge, ICP were recovered from the bottom of the centrifuge tubes. Isolated ICP were then washed with TBS until little or no cholesterol was released. Most (>85%) of cholesterol in the dialyzed d=1.02-1.07 g/ml density fraction was associated with ICP. Few or no ICP was recoverable in the d=1.02-1.07 g/ml density region when normal aortas were subjected to the above isolation steps. The composition of isolated ICP was determined by normalizing the masses of proteins, PL, total and unesterified cholesterol and triglycerides associated with ICP by total mass, on a per ICP preparation basis.

### Preparation of Plasma and Blood

Normolipidemic plasma was obtained from the Alabama Regional Blood Center, Birmingham, Ala. and from volunteers. To obtain blood with a high density of red blood cell (RBC), fresh blood samples were subjected to low speed centrifugation (1000 rpm for 10 min) to separate ⅔ of plasma from RBC and to entrap the remaining plasma within packed RBC. Samples of plasma and packed RBC were used. In some experiments, sterile R-HDL was added to blood (2 mg R-HDL PC/ml blood) prior to centrifugation, and this supplemented plasma was used.

### Preparation of native and modified HDL, apo HDL, R-HDL, and Lipid Vesicles

HDL was isolated from normolipidemic plasma by the sequential flotation method (27) and washed once by re-isolating it by density gradient ultracentrifugation (26). Apo HDL was prepared by delipidating isolated, lyophilized HDL with an ethanol: either mixture (3:2, v:v) mixture (28). Discoidal R-HDL was prepared by mixing apo HDL with unilamellar DMPC liposomes at weight (mass) ratios of 1:2, 1:4 or 1:8 followed by sonication. R-HDL containing cholesterol was prepared from apo HDL and DMPC containing 10% FC. R-HDL was also prepared from egg PC by solubilizing apo HDL and egg PC in sodium cholate and subsequently removing excess sodium cholate by dialysis (29). Prepared R-HDL was optically clear, and apo A-I and PC of R-HDL were recovered as a single HDL-density peak in the density gradient tube. Multilamellar DMPC vesicles and SPM vesicles enriched with FC (SPM/FC mass ratio=1) or lacking cholesterol were prepared by evaporating the organic solvent containing DMPC and SPM±FC, and swelling the dried lipids in TBS. These multilamellar vesicles are readily pelletable by centrifugation at 10,000 rpm for 15 min in a microcentrifuge. Unilamellar DMPC liposomes were also prepared by sonicating the multilamellar DMPC vesicles and subsequently removing multilamellar vesicles, if any, from the sonicated sample by centrifugation. To prepare DMPC-enriched HDL, fresh HDL was incubated with an excess amount of multilamellar DMPC vesicles at room temperature (24° C.) for 4 hr. Undisrupted multilamellar DMPC vesicles were removed (pelleted) by centrifugation, leaving optically clear DMPC-enriched HDL. PL to protein ratios of DMPC-enriched HDL and untreated HDL were then determined by measuring the levels of PL and total proteins.

### HDL-, apo HDL-, R-HDL-, PC Liposome- and/or Plasma-mediated Release of Cholesterol from ICP and Change in Level and Composition of Lipids and/or Number of Cholesterol Crystals of ICP

The overall experimental paradigm involved 1) incubating ICP (0.5-1.0 mg FC) with TBS, TBS-containing HDL, apo HDL, R-HDL, PC liposomes or plasma in a 1.5 ml-size conical tube at 37° C. for 2-16 hrs, 2) separating (pelleting) undissolved ICP by centrifugation at 10,000 rpm in a microcentrifuge for 15 min at room temperature, 3) measuring the levels of cholesterol or PL released from ICP into incubation medium, 4) determining changes in the level and/or composition of PL on ICP, HDL, and R-HDL and 5) determining the number of cholesterol crystals on control and treated ICP. Levels of cholesterol and PL released from ICP into incubating medium were measured by a Technicon Autoanalyzer using enzymatic assay reagent for cholesterol and PL. PL composition of ICP, HDL, and R-HDL were examined following extraction of their lipids with a chloroform: methanol (2:1, v/v) mixture (30) by silica gel thin-layer chromatography (TLC) using chloroform: methanol: ammonium hydroxide (65:25: 4) mixtures as a developing solvent and densitometric scanning of the developed TLC plate using an auto scanner (Helena Laboratory, Beaumont, Tex.). This scanner normalizes the density of each individual band on the TLC plate to the maximum density (darkest band) in the TLC plate. In experiments comparing the potency of fresh plasma to release cholesterol from ICP and RBC, fresh plasma processing active lecithin cholesterol acyltransferase (LCAT) and cholesterylester (CE) transfer proteins (CETP) was incubated with ICP or RBC (1 ml packed RBC contain 1.3 mg cholesterol (31) at a plasma total cholesterol to ICP FC or RBC FC ratio of approximately 1:1. Changes in plasma cholesterol level and/or distribution of cholesterol among lipoproteins were determined by the by the lipoprotein cholesterol auto-profiler method (26). This method involves the rapid separation of lipoproteins and lipoprotein-like particles in different density ranges by short spin density gradient ultracentrifugation (50,000 rpm for 150 min), continuous online mixing of the effluent from density gradient tubes with a cholesterol enzymatic reagent, and online measurement of absorbance.

Change in numbers of cholesterol crystals on control ICP following incubation with apo HDL, HDL or R-HDL were examined by a polarizing microscope. Briefly, 30 μl aliquots of control and treated ICP were placed on clean glass slides, allowed to dry for 1 hr, and examined using a Nikon Optiphot 2 microscope with a polarizing filter. Images were captured using a Sensicam CCD camera (Cooke Instruments) and IP Lab software (Scanalytics, Fairfax Va.). For quantitative studies, dried aliquots were sampled systematically to produce 10 images for each droplet. The number and size of cholesterol crystals were determined from digital images using the UVP Lab Work colony counting computer program (Quest Scientific Co. Cumming, Ga.). The percent decrease in the number of cholesterol crystals on control ICP following incubation with apo HDL, HDL or R-HDL was determined

### Characterization of Soluble Cholesterol-containing Particles Formed from Insoluble ICP by R-HDL, apo HDL or Plasma

Soluble supernatant fractions separated from incubation mixtures of ICP and R-HDL were characterized by enzymatic assay for PL and cholesterol levels, TLC for PC to SPM ratio, and by density gradient ultracentrifugation for the density spectrum of cholesterol, PL, and apo A-I. The density spectrums of cholesterol, PL and apo A-I on control and ICP-treated R-HDL were examined by lipoprotein cholesterol auto-profiler method (26) described above and by determining the distribution of PL and apo A-I among density gradient fractions using an apo A-I immunoassay assay kit and enzymatic PL assay kit.

### R-HDL Interaction with Artificial Mutilamellar SPM Vesicles Rich or Lacking in FC

To determine whether an abnormally high level of SPM and FC on ICP is a factor that allows the transfer of PC from HDL or R-HDL to ICP, we examined the transfer of PC from R-HDL (apo HDL: DMPC=1: 4) to multilamellar SPM vesicles enriched with FC (SPM/FC mass ratio=1) or lacking cholesterol. Briefly, pelleted multilamellar SPM vesicles with or without FC were incubated with R-HDL at a SPM to R-HDL PC ratio of 1:5 for 6 hr at 37° C. Following incubation, undisrupted multilamellar SPM vesicles were pelleted by centrifugation at 10,000 rpm for 15 min and washed 3× with TBS. The PL composition of the upper soluble fraction and pelleted multilamellar SPM vesicles was determined by the TLC method as described above.

### Statistical Analysis

Quantitative variables were expressed as mean±S.D. The Tukey's multiple comparisons test was used to determine which means are significantly different among different treatment groups, while the paired t-test was used to determine any difference in specific pairs of means. All statistical tests were two-sided and performed at a 5% significance level. All statistical analyses were performed with the use of SAS software (version 8.2), SAS Institute Inc., Cary, N.C.

### Animal Study

Male New Zealand White rabbits (n=32), 8-weeks old (4-5 lbs) were housed in stainless steel cages, and all studies were performed under the approval of the Instititional Animal Care and Use Committee on animal investigations. Rabbits in the control group (=12) were maintained on a normal rabbit chow diet for the full 15-week period. To induce the development of atherosclerosis, animals (n=24) were fed with atherogenic diet containing 2% cholesterol and 6% peanut oil for 6 weeks. The feed consumption decreased, and the diet was changed to the normal rabbit chow for 4 weeks. During this period, 11 animals died due to liver failure with massive fatty liver in cholesterol-fed group. At the tenth week, animals in the cholesterol-fed group (n=13) were divided into two subgroups, a treatment group (Group I) and a no treatment group (Group II). Animals in Group I (n=5) were injected with sterilized saline solution as a control, using a butterfly catheter via ear vein. Animals in the Group I (n=8) were injected with 5 ml of DMPC liposomes (300 mg/kg body weight) weekly for 5 weeks (5 times in 35 day period) using the same procedure. During this 5-week period, rabbits in both Groups I and II received normal rabbit chow; in addition the control group rabbits were also maintained on normal rabbit chow for an additional 5 weeks.

### Non-standard Abbreviations Used

Apo, apolipoproteins; CE, cholesterylester; CETP, cholesterylester transfer proteins; DMPC, dimyristoyl phosphatidylcholine; FC, free cholesterol; ICP: insoluble component of plaques; LCAT, lecithin:cholesterol acyltransferase; PC, phosphatidylcholine; PL, phospholipids; RBC, red blood cells, RCT, reverse cholesterol transport; R-HDL; reconstituted HDL; SPM, sphingomyelin; TBS, Tris-buffered saline; TLC, thin-layer chromatography.

### REFERENCES

1. G. J. Gordon, J. L. Probstfeld, R. J. Garrisom, J. D. Neaton, W. P. Castelli, J. D. Knoke, J. R. Jacobs, S. Bangdlwala, A. Tyroler, Circulation. High density lipoprotein cholesterol and cardiovascular disease. Four prospective American studies. Circulation 79 (1989) 8-15.
2. P. Puchois, A. Kandossi, 0. Fievr, J. L. Fourrier, M. Bertland, E. Koren, J. C. Fruchard, Apolipoprotein A-I containing lipoproteins in coronary heart disease. Atherosclerosis 68 (1987) 35-46.
3. T. Gordon, W. P. Castelli, M. Hjortland, W. Kannel, T. Dawber, High density lipoprotein as a protective factor against coronary heart disease. Am. J. Med. 62 (1977) 707-714.
4. J. A. Glomset, The plasma lecithin:cholesterol acyltransferase. J. Lipid Res. 9 (1968) 155-167
5. Phillips, M. C., W. J. Johnson, and G. H. Rothblat. Mechanism and consequence of cellular cholesterol exchange and transfer. Biochim. Biophys. Acta. 906 (1987) 223-276.
6. G. H. Rothblat, M. D. L. Llera-Moya, V. Atger, G. Kellner-Weibel, D. L. William, M. C. Philips, Cell cholesterol efflux: integration of old and new observations provides new insights. J. Lipid Res. 40 (1999) 781-796.
7. Jian, B., M. de la Llera Moya, Y. Ji, N. Wang, M. C. Phillips, J. B. Swaney, A. R. Tall, and G. H. Rothblat. Scavenger receptor class B type I as a mediator of cellular cholesterol efflux to lipoproteins and phospholipid acceptors. J. Biol. Chem. 273 (1998) 5599-5606.
8. de la Llera-Moya, M., G. H. Rothblat, M. A. Connelly, G. Kellner-Weibel, S. W. Sakr, M. C. Phillips, and D. L. Williams. Scavenger receptor BI (SR-BI) mediates free cholesterol flux independently of HDL tethering to the cell surface. J. Lipid Res. 40 (1999) 575-580.
9. Lawn, R. M., D. P. Wade, M. R. Garvin, X. Wang, K. Schwartz, J. G. Porter, J. J. Seilhamer, A. M. Vaughan, and J. F. Oram. 1999. The Tangier disease gene product ABC1 controls the cellular apolipoprotein-mediated lipid removal pathway. J. Clin. Invest. 104 (1999) R25-R31.
10. Brousseau, M. E., G. P. Eberhart, J. Dupuis, B. F. Asztalos, A. L. Goldkamp, E. J. Schaefer, and M. W. Freeman. 2000. Cellular cholesterol efflux in heterozygotes for Tangier disease is markedly reduced and correlates with high density lipoprotein cholesterol concentration and particle size. J. Lipid Res. 41 (2000) 1125-1135.
11. K. L. Gillotte, M. Zaiou, L. Lund-Katz, G. M. Anatharamaiah, P. Holvoet, A. Dhoest, M. N. Palgunachari, J. P. Segrest, K. H. Weisgraber, G. H. Rothblat, M. C. Phillips, Apolipoprotein-mediated plasma membrane microsolubilization. Role of lipid affinity and membrane penetration in the efflux of cellular cholesterol and phospholipid. J. Biol. Chem. 274 (1999) 2021-2028.
12. D. M. Small, Progression and regression of atherosclerotic lesions. Insight from lipid physical biochemistry. Arteriosclerosis 8 (1988) 103-129.
13. H. Shio, H. J. Haley, S. Fowler, Characterization of lipid-ladden aortic cells from cholesterol-fed rabbits. Lab. Invest. 39 (1978) 390-397.
14. J. R. Guyton, K. F. Klemp, The lipid-rich core region of human atheroclerotic fibrous plaques: prevalence of small lipid droplets and vesicles by electron microscopy. Am. J. Pathol. 134 (1989) 705-717.
15. H. S. Kruth, Subendothelial accumulation of unesterified cholesterol: an early event in atherosclerotic lesion development. Atherosclerosis 57 (1985) 337-341.
16. F. F. Chao, J. Blanchette-Mackie, B. F. Dickens, W. Gamble, H. S. Kruth, Development of unesterified cholesterol-rich lipid particles in atherosclerotic lesions of WHHL and cholesterol fed NZW rabbits. J. Lipid Res. 35 (1995) 71-83.
17. I. Tabas, Review. Cholesterol and phospholipid metabolism in macrophages. Biochim. Biophys. Acta 1529 (2000) 164-174.
18. R. Y. Ball, E. C. Stowers, J. H. Burton, N. R. B. Cary, J. N. Skepper, M. J. Michinson, Evidence that the death of macrophage foam cells contributes to the lipid core of atheroma. Atherosclerosis 114 (1995) 45-54.
19. S. K. Seth, H. A. I. Newman, Sphingomylein and other phospholipid metabolism in the rabbit atheromatous and normal aorta. Circ. Res. 36 (1975) 294-299.
20. S. N. Jagannathan, W. E. Connor, W. H. Baker, A. K. Bhattacharya, The turnover of cholesterol in human atherosclerotic arteries. J. Clin. Invest. 54 (1974) 366-377.
21. S. S. Katz, D. M. Small, F. R. Smith, R. D. Dell, D. S. Goodman, Cholesterol turnover in lipid phases of human atherosclerotic plaques. J. Lipid Res. 23 (1982) 733-737.
22. Y. H. Abdulla, C. W. M. Adams, The action of human high density lipoprotein on cholesterol crystals. Part 1. Light-microscopic observations. Atherosclerosis 31 (1978) 465-471.
23. Y. H. Abdulla, C. W. M. Adams, The action of human high density lipoprotein on cholesterol crystals. Part 2. Biochemical observations. Atherosclerosis 31 (1978) 473-480.
24. B. H. Chung, G. Tallis, V. Yalamoori, G. M. Anantharamaiah, J. P. Segrest, Liposome-like particles isolated from human atherosclerotic plaques are structurally and compositionally similar to surface remnants of triglyceride-rich lipoproteins. Arterioscler. Thromb. Vasc. Biol. 14 (1994) 622-635.
25. S. S. Katz, D. M. Small, Isolation and partial characterization of the lipid phases of human atherosclerotic plaques. J Biol. Chem. 255 (1980) 9753-9759.
26. B. H. Chung, J. P. Segrest, M. L. Ray, J. D. Brunzell, J. E. Hokanson, R. M. Krauss, K. Beaudrie, J. T. Cone, Single vertical spin density gradient ultracentrifugation. Method in Enzymol. 128 (1986) 81-209.
27. R. J. Havel, H. A. Eder, J. H. Bragdon, The distribution and chemical composition of ultracentrifugally separated lipoproteins in human serum. J. Clin. Invest. 34 (1955) 1345-1353.
28. A. M. Scanu, C. Edelstein, Solubility in aqueous solutions of ethanol of the small molecular weight peptides of the serum very low density and high density lipoproteins: relevance to recovery problem during delipidation of serum lipoproteins. Anal. Biochem. 44 (1971) 576-588.
29. Jonas A. Reconstitution of high density lipoproteins. Method of Enzymol 128 (1986) 553-582.
30. J. Folch, M. Lees, C. M. Sloane-Stanley, A simple method for isolation and purification of total lipids from animal tissues. J. Biol. Chem. 226 (1957) 497-509
31. J. T. Dodge, C. Mitchell, D. Hanahan. The preparation and chemical characteristics of hemoglobin-free ghosts of human erythrocytes. Arch. Biochem. Biophys. 100 (1963) 119-30.
32. B. H. Chung, F. Franklin, S. B. H. Cho, J. P. Segrest, K. Hart, B. E. Darnell. Potencies of lipoproteins in fasting and postprandial plasma to accept additional cholesterol molecules released from cell membranes. Arterioscler, Thromb. Vasc. Biol. 18 (1998) 1217-1230.
33. R. A. Demel, J. W. C. M Jansen, P. W. M. Van Dijik, L. L. M. Van Deenen, The preferential interaction of cholesterol with different classes of phospholipids. Biochim. Biophys. Acta. 165 (1977) 1-10.
34. A. V. Chobanian, W. Hollander, Body cholesterol metabolism in man: the equilibrium of serum and tissue cholesterol. J. Clin. Invest. 41 (1962) 1732-1737.
35. Y. K. Ho, M. S. Brown, J. L. Goldstein, Hydrolysis and excretion of cytoplasmic cholesterylesters by macrophages: stimulation by high density lipoprotein and other agent. J. Lipid Res. 21 (1980) 391-398.
36. B. J. O'Connell, M. Denis, J. Genest. Cellular physiology of cholesterol efflux in vascular endothelial cells. Circulation. 110 (2004) 2882-2888.
37. K. J. Williams, V. P. Werth, J. A. Wolff. Intravenously administered lecithin liposomes: a synthetic antiatherogenic lipid particle. Perspect. Biol. Med. 27 (1984) 417-431.
38. K. J. Williams, S. Vallabhajosula, I. U. Rahman, T. M. Donnelly, T. S. Parker, M. Weinrauch, S. J. Goldsmith. Low density lipoprotein receptor-independent hepatic uptake of a synthetic, cholesterol-scavenging lipoprotein: implications for the treatment of receptor-deficient atherosclerosis. Proc. Natl. Acad. Sci. USA. 85(1988) 242-246.
39. J. B. Massey, D. Hickson, H. S. She, J. T. Sparrow, D. P. Via, A. M. Gotto, H. J. Pownall, Measurement and prediction of the rate of spontaneous transfer of phospholipids between plasma lipoproteins. Biochim. Biophys. Acta. 794 (1984) 274-280.
40. A. R. Tall, S. Krumholz, T. Olivercrona, R. J. Deckelbaum. Plasma phospholipid transfer protein enhances transfer and exchange of phospholipids between very low density lipoproteins and high density lipoproteins during lipolysis. J. Lipid Res. 26 (1985) 842-851.
41. A. T. Remaley, B. D. Farsi, A. C. Shirali, J. M. Hoeg, H. B. Brewer, Differential rate of cholesterol efflux from the apical and basolateral membranes of MDCK cells. J. Lipid Res. 39 (1998) 1231-1238.
42. J. C. Gold, M. C. Phillips, Effect of membrane lipids and proteins and cytoskeletal proteins on the kinetics of cholesterol exchange between high density lipoproteins and human red blood cells, ghosts and microvesicles. Biochim. Biophys. Acta. 1111 (1992) 103-110.
43. J. P. Slotte, J. Tenhunens, I. Porn, Effect of sphingomyelin degradation on cholesterol mobilization and efflux to high density lipoproteins in cultured fibroblasts. Biochim. Biophys. Acta. 1025 (1990) 152-156.
44. W. V. Rodrigueza, K. J. Williams, G. H. Rothblat, M. C. Phillips, Remodeling and shuttling. Mechanism for synergistic effects between different acceptor particles in the mobilization of cellular cholesterol. Arterioscler. Thromb. Vasc. Biol. 17 (1997) 383-393.
45. R. W. Clark, T. A. Sutfin, R. B. Ruggeri, A. T. Willauer, E. D. Sugarman, G. Magnus-Aryitey, P. G. Cosgrove, T. M. Sand, R. T. Wester, J. A. Williams, M. E. Perlman, M. J. Bamberger. Raising high-density lipoproteins in humans through inhibition of cholesteryl ester transfer protein: An initial multidose study of Torectrapib. Arterioscler. Thromb. Vasc. Biol. 24 (2004) 490-497.
46. M. Ishigami, S. Yamashita, N. Sakai, T. Arai, K. Hirano, H. Hiraoka, K. Kameda-Takemura, Y. Matsuzawa. Large and cholesteryl ester-rich high-density lipoproteins in cholesteryl ester transfer protein (CETP) deficiency cannot protect macrophages from cholesterol accumulation induced by acetylated low-density lipoproteins. J Biochem. 116 (1994) 257-262.
47. S. Yamashita, T. Maruyama, K. Hirano, N. Sakai, N. Nakajima, Y. Matsuzawa. Molecular mechanisms, lipoprotein abnormalities and atherogenicity of hyperalphalipoproteinemia. Atherosclerosis. 152 (2000) 271-285.
48. P. K. Shah, J. Yano, 0. Reyes, K-U. Chyu, S. Kaul, C. L. Bisgaier, S. Drake, B. Cercek, High-dose recombinant apolipoprotein A-I Milano mobilizes tissue cholesterol and rapidly reduces plaque lipid and macrophage content in apolipoprotein E deficient mice. Circulation 103 (2001) 3047-3050.
49. K. J. Williams, R. Scalia, K. D. Mazany, W. V. Rodrigueza, A. M. Lefer. Rapid restoration of normal endothelial functions in genetically hyperlipidemic mice by a synthetic mediator of reverse lipid transport. Arterioscler. Thromb. Vasc. Biol. 20 (2000) 1033-1039,
50. S. E. Nissen, T. Tsunoda, E. M. Tuzcu, P. Schoenhagen, C. J. Cooper, M. Yasin, G. M. Eaton, M. A. Kauer, W. S. Sheldon, C. L. Grines, S, Halpern, T. Crowe, J. C. Blankenship, R. Kerensky, Effect of recombinant apo A-I Milano on coronary atherosclerosis in patients with acute coronary syndromes. A randomized controlled trial. JAMA 290 (2003) 2292-2230.
51. 0. Stein, Y. Stein, Atheroprotective mechanisms of HDL. Atherosclerosis 144 (1999) 285-301.

What is claimed:

1. A method for reducing the volume of a plaque in a subject, said method comprising the step of administering to said subject a therapeutically effective amount of a lipid composition comprising phosphatidylcholine, said phosphatidylcholine being dimyristoyl phosphatidylcholine or egg phosphatidylcholine, whereby at least a portion of said phosphatidylcholine is transferred from said lipid composition to said plaque.

2. A method for increasing the stability of a plaque in a subject, said method comprising the step of administering to said subject a therapeutically effective amount of a lipid composition comprising phosphatidylcholine, said phosphatidylcholine being dimyristoyl phosphatidylcholine or egg phosphatidylcholine, whereby at least a portion of said phosphatidylcholine is transferred from said lipid composition to said plaque.

3. A method of increasing the percentage of a phospholipid component in a plaque in a subject, said method comprising the step of administering to said subject a therapeutically effective amount of a lipid composition comprising phosphatidylcholine, said phosphatidylcholine being dimyristoyl phosphatidylcholine or egg phosphatidylcholine, whereby at least a portion of said phosphatidylcholine is transferred from said lipid composition to said plaque.

4. A method for enriching a plaque in a subject with phosphatidylcholine, said method comprising the step of administering to said subject a therapeutically effective amount of a lipid composition comprising phosphatidylcholine, said phosphatidylcholine being dimyristoyl phosphatidylcholine or egg phosphatidylcholine, whereby at least a portion of said phosphatidylcholine is transferred from said lipid composition to said plaque.

5. A method for decreasing a free cholesterol to phospholipid ratio in a plaque from a subject, said method comprising the step of administering to said subject a therapeutically effective amount of a lipid composition comprising phosphatidylcholine, said phosphatidylcholine being dimyristoyl phosphatidylcholine or egg phosphatidylcholine, whereby at least a portion of said phosphatidylcholine is transferred from said lipid composition to said plaque.

6. (canceled)

7. (canceled)

8. The method of any of claims 1-5 where said lipid composition further comprises a protein component.

9. The method of claim 9 where said protein component is an apolipoprotein.

10. The method of claim 10 where said apolipoprotein is apolipoprotein A1.

11. The method of claim 10 where said apolipoprotein to phosphatidylcholine ration is from about 1:1 to about 1:8.

12. The method of any of claims 1-5 where said lipid composition is a high density lipoprotein.

13. (canceled)

14. (canceled)

15. The method of any of claims 1-5 where said lipid composition is a liposome.

16. (canceled)

17. (canceled)

18. The method of any of claims 1-5 where said subject is a human.

19. A method for releasing cholesterol from a plaque in a subject, said method comprising the steps of administering to said subject a therapeutically effective amount of a lipid composition comprising phosphatidylcholine, said phosphatidylcholine being dimyristoyl phosphatidylcholine or egg phosphatidylcholine, whereby at least a portion of said phosphatidylcholine is transferred from said lipid composition to said plaque, and further administering to said subject a therapeutically effective amount of a cholesterol mobilizing agent whereby at least a portion of the cholesterol component of the plaque is transferred from said plaque to said cholesterol mobilizing agent.

20. The method of claim 19 where the cholesterol mobilizing agent is fresh plasma or a second lipid composition not comprising phosphatidylcholine.

21. The method of claim 19 where said second lipid composition comprises at least one protein.

22. The method of claim 21 where said at least one protein is an apolipoprotein.

23. The method of claim 22 where said apolipoprotein is apolipoprotein A1.

24. The method of claim 23 where said apolipoprotein to phospholipid ratio is from about 1:1 to about 1:8.

25. (canceled)

26. (canceled)

27. The method of any of claims 19-24 where said lipid composition is a high density lipoprotein.

28. (canceled)

29. (canceled)

30. The method of any of claims 19-24 where said lipid composition is a liposome.

31. (canceled)

32. (canceled)

33. The method of any of claims 19-24 where said subject is a human.

34. A method for releasing cholesterol from a plaque in a subject, said method comprising altering the lipid composition of said plaque in the subject by administering to said subject a therapeutically effective amount of a lipid composition comprising phosphatidylcholine, whereby at least a portion of said phosphatidylcholine is transferred from said lipid composition comprising phosphatidylcholine to said plaque, and further administering to said subject a therapeutically effective amount of a cholesterol mobilizing agent whereby at least a portion of the cholesterol component of the plaque is transferred from said plaque to said cholesterol mobilizing agent.

35. The method of claim 34 where said phosphatidylcholine is dimyristoyl phosphatidylcholine or egg phosphatidylcholine.

36. The method of claim 34 where the cholesterol mobilizing agent is fresh plasma or a second lipid composition not comprising phosphatidylcholine.

37. The method of claim 34 where said second lipid composition comprises at least one protein.

38. The method of claim 37 where said at least one protein is an apolipoprotein.

39. The method of claim 37 where said apolipoprotein is apolipoprotein A1.

40. The method of claim 39 where said apolipoprotein to phospholipid ratio is from about 1:1 to about 1:8.

41. A method for increasing the stability of a plaque in a subject or regressing said plaque said subject, said method comprising altering the lipid composition of said plaque in the subject by administering to said subject a therapeutically effective amount of a lipid composition, whereby at least a portion of said phosphatidylcholine is transferred from said lipid composition to said plaque.

42. The method of claim 41 where said phosphatidylcholine is dimyristoyl phosphatidylcholine or egg phosphatidylcholine.

43. The method of claim 41 where said lipid composition is a high density lipoprotein or a liposome.

44. The method of claim 41 where said lipid composition further comprises a protein component.

45. The method of claim 44 where said protein component is an apolipoprotein.

46. The method of claim 44 where said apolipoprotein is apolipoprotein A1.

47. The method of claim 46 where said apolipoprotein to phosphatidylcholine ration is from about 1:1 to about 1:8.
